(19) United States
(12) Patent Application Publication
(10) Pub. No.: US 2026/0139230 A1
(43) Pub. Date: May 21, 2026

(54) METHOD AND SYSTEM FOR MULTI-CELLULAR BIOPRINTING OF VASCULARIZED TISSUE CONSTRUCTS

(71) Applicants: City University of Hong Kong, Hong Kong (HK); The University of Hong Kong, Hong Kong (HK)

(72) Inventors: Jian LU, Hong Kong (HK); Kwan MAN, Hong Kong (HK); Wanying WANG, Hong Kong (HK); Rongrong ZHONG, Hong Kong (HK); Jiang LIU, Hong Kong (HK)

(21) Appl. No.: 19/369,313

(22) Filed: Oct. 26, 2025

Related U.S. Application Data
(60) Provisional application No. 63/722,558, filed on Nov. 19, 2024.

(51) Int. Cl. C12N 5/071; B33Y 10/00; B33Y 70/00; B33Y 80/00

(52) U.S. Cl. CPC C12N 5/0697; B33Y 10/00; B33Y 70/00; B33Y 80/00; C12N 2502/1352; C12N 2502/28; C12N 2513/00; C12N 2533/54; C12N 2537/10; C12N 2539/00

(57) ABSTRACT

The present invention relates to systems, compositions, and methods for multi-cellular bioprinting of vascularized tissue constructs comprising hierarchical vascular networks. The method employs a dual-bioink strategy involving a fibrous microgel matrix bioink with tunable stiffness, self-healing rheology, and microporosity to support capillary self-assembly, and a sacrificial bioink that defines perfusable macro-channels upon removal. The system integrates top-down printing with bottom-up cellular self-organization within a programmable bioprinting platform. A key inventive aspect lies in engineering the physical topography of the matrix to actively guide vascular morphogenesis, moving beyond conventional permissive scaffolds. The integrated platform enables the fabrication of complex, biomimetic vascular networks with enhanced mass transport, cell viability, and in vivo integration potential. The invention represents a significant advance toward scalable production of functional human tissues, with applications in regenerative medicine, disease modeling, and drug discovery.

### CROSS-REFERENCE TO RELATED APPLICATIONS

The present application claims the priorities from the U.S. provisional patent application Ser. No. 63/722,558 filed Nov. 19, 2024, and the disclosure of which is incorporated herein by reference in its entirety.

### FIELD OF THE INVENTION

The present invention generally relates to at least the fields of regenerative medicine and tissue engineering, bioprinting technologies, drug discovery, and so forth.

### BACKGROUND OF THE INVENTION

Engineered tissues with functional, multiscale vascular networks are essential for addressing major challenges in regenerative medicine. In native organs, a hierarchical vascular system composed of large vessels, microvessels, and capillaries ensures effective oxygen and nutrient transport, waste removal, and dynamic signaling. Without such networks, tissue constructs exceeding a few hundred micrometers in thickness often experience hypoxia, leading to necrosis and loss of function. To recreate these physiological features, engineered tissues must incorporate both perfusable macrovascular structures and capillary-like microvessels in a spatially organized and scalable manner. These multiscale systems are vital not only for transplantable tissue grafts but also for in vitro platforms used in drug discovery, ischemia modeling, and organ-specific disease studies.

Existing vascularization strategies typically fall into two categories: top-down and bottom-up approaches. Top-down techniques, such as extrusion-based 3D bioprinting, allow precise patterning of perfusable channels and are effective for replicating macro-scale architectures. However, the use of high-concentration bulk hydrogels often limits cell motility and matrix remodeling, impairing the formation of native-like microvasculature. Bottom-up approaches, such as organoid formation or self-assembly within permissive hydrogels, support microvascular development but generally lack structural control, scalability, and perfusability.

Current 3D printed vascular models derived from imaging data may replicate the geometry of macrovessels with high fidelity, but fail to recreate capillary sprouting or microvascular complexity. Conversely, vascular organoids offer microvascular mimicry, yet are embedded in amorphous matrices such as Matrigel that lack mechanical strength and are incompatible with scalable tissue fabrication. These limitations are especially evident in liver tissue engineering, where current artificial constructs often fail to replicate the sinusoidal architecture of native liver vasculature. As a result, hepatocyte-laden hydrogels commonly suffer from poor perfusion, central necrosis, and short-term viability.

Several prior art references illustrate partial progress in this area but do not fully address the challenge of scalable, multi-scale vascularization. For example, U.S. Patent Application Publication No. US20200289709 A1 discloses tissue constructs using undefined “particulate-like tissue” and general “ink” formulations, but does not describe a dual-bioink strategy or specific methods for forming hierarchical vascular structures. While it mentions perfusable networks, the document lacks clarity on how microvessels and capillaries are generated, and no in vivo evaluation is provided to demonstrate integration potential. Similarly, U.S. Patent Application Publication No. US20180313822 A1 focuses on engineered tissues for in vitro use and describes pseudo-vascular networks, but does not emphasize multi-scale vascular architecture or offer approaches for combining macro- and microvessels in a coherent system. Furthermore, the methods are limited to in vitro contexts, with no suggestion of host integration or in vivo applicability. Despite these advances, no existing strategy has successfully integrated architectural precision and microvascular self-organization within a single, scalable platform. As such, current technologies remain inadequate for recapitulating the structural and functional complexity of native vascularized tissues.

Therefore, there is a need in the art for a unified vascularization strategy that enables the scalable formation of multiscale, perfusable, and tissue-specific vascular networks, overcoming the structural and functional limitations of current approaches.

### SUMMARY OF THE INVENTION

The present invention aims to address a fundamental challenge in tissue engineering: the scalable fabrication of thick, vascularized tissues that faithfully recapitulate the hierarchical vascular architecture of native organs. In particular, the invention seeks to overcome the limitations of existing top-down and bottom-up biofabrication methods, which have thus far failed to provide an integrated platform capable of generating both perfusable macrovascular channels and biologically self-organized microvascular networks within a single tissue construct.

The present invention provides an integrated biomanufacturing platform for fabricating engineered tissue constructs comprising hierarchical vascular networks. It addresses a fundamental limitation in tissue engineering—namely, the inability to generate thick, metabolically active tissues that include both perfusable macro-scale channels and biologically relevant microvascular structures. The invention overcomes this challenge through a synergistic combination of a novel fibrous microgel bioink and multiple advanced bioprinting architectures.

The fibrous microgel bioink introduced herein possesses a unique set of properties designed to support vascular morphogenesis and enable complex tissue fabrication. First, it comprises a microporous architecture formed by discrete micro-units, yielding a highly permeable network that supports mass transport and cellular migration. Second, it exhibits thixotropic and self-healing rheology, allowing the bioink to function both as a printable matrix and as a support bath for embedded or suspension printing. Third, its mechanical stiffness can be tuned via photopolymerization to match native tissue stiffness (e.g., liver). Fourth, and most critically, the micro-topographical features of the fibrous network provide structural cues that guide vascular cells to self-organize into tissue-specific capillary architectures such as sinusoidal networks. These capabilities distinguish the fibrous microgel from conventional bulk hydrogels, which lack permeability, responsiveness, or biomimetic structure.

The method employs a dual-bioink strategy, wherein the matrix bioink (e.g., fibrous microgel with encapsulated cells) is printed alongside a sacrificial bioink that can be selectively removed to form open, perfusable macrochannels. In certain embodiments, the sacrificial ink may be cell-laden and time-delayed in dissolution, allowing the released endothelial cells to adhere to channel surfaces and form luminal linings. In this manner, the invention integrates architectural precision with biological self-organization within a single fabrication workflow.

In one aspect, the present invention provides a method for multi-cellular bioprinting of vascularized tissue constructs, including preparing a degradable matrix bioink comprising gelatin methacrylate (GelMA) and gelatin; preparing a sacrificial bioink comprising a hydrogel-forming component and a calcium agent; loading the matrix bioink and the sacrificial bioink into separate cartridges of a multi-nozzle three dimensional (3D) bioprinter; co-printing the matrix bioink and the sacrificial bioink in an alternating or embedded pattern to form a 3D tissue construct; crosslinking the matrix bioink and removing the sacrificial bioink to create perfusable channels within the construct; and forming the vascularized tissue constructs comprising a degradable matrix and an integrated network of perfusable channels.

In accordance with one embodiment, the matrix bioink further includes alginate.

In accordance with one embodiment, the hydrogel-forming component includes gelatin, hyaluronic acid, agarose, alginate, gellan gum, collagen, polyethylene glycol (PEG)-based polymers, or a combination thereof.

In accordance with one embodiment, the calcium agent includes calcium chloride, calcium sulfate, or a combination thereof.

In accordance with one embodiment, the matrix bioink includes microporous fibrous microgel particles formed by fragmenting a crosslinked bulk hydrogel, and the fibrous microgel particles are prepared by extruding a crosslinked hydrogel through a 20 μm mesh.

In accordance with one embodiment, the sacrificial bioink includes endothelial cells at a density in a range from 5×10⁵ to 2×10⁶ cells/mL, and wherein the cells adhere to the inner channel surfaces after sacrificial ink removal.

In accordance with one embodiment, the 3D tissue construct includes a hexagonal macrochannel geometry mimicking native liver lobules.

In accordance with one embodiment, the sacrificial bioink is printed directly into a self-healing support bath comprising a fibrous microgel jammed microporous matrix. The support bath self-heals around the printed filaments prior to crosslinking, and comprises a first population of cells such that the resulting three-dimensional tissue construct exhibits hierarchical porosity defined by the printed macrochannels and intrinsic microporosity, and supports capillary self-assembly within the matrix.

In accordance with one embodiment, the printed macrochannels define a perfusable circuit comprising at least one inlet and at least one outlet.

In accordance with one embodiment, the sacrificial bioink is removed post-printing by thermal liquefaction or enzymatic degradation.

In accordance with one embodiment, the crosslinking step is performed via photocrosslinking and/or ionic crosslinking.

In another aspect, the present invention provides a vascularized tissue construct for implantation into a host organ. The vascularized tissue construct includes a microporous matrix with interconnected micropores formed from a bioink composition, a perfusable network of macrochannels extending through the microporous matrix, and one or more populations of living cells encapsulated within the microporous matrix. The vascularized tissue construct exhibits hierarchical porosity defined by the macrochannels and the interconnected micropores of the microporous matrix. The construct comprises a self-organized microvascular plexus formed within the microporous matrix.

In accordance with one embodiment, the bioink is formed by (a) forming a pre-gel solution containing GelMA, gelatin, and LAP; (b) photocrosslinking the solution to form a bulk hydrogel; (c) fragmenting the hydrogel through a mesh to form microparticles; (d) suspending the microparticles in phosphate-buffered saline; and (e) subjecting the suspension to centrifugation at 10,000 rpm for 5 minutes to remove excess liquid, thereby obtaining the final printable bioink.

In accordance with one embodiment, the bioink composition includes a suspension of crosslinked hydrogel microparticles comprising GelMA and gelatin, wherein the suspension of crosslinked hydrogel microparticles form a jammed, microporous matrix with interconnected pores ranging from 20 to 50 μm, and the matrix exhibits shear-thinning and self-healing thixotropic properties, and provides topographical guidance to support the formation of a tissue-specific architecture.

In accordance with one embodiment, the interconnected micropores are configured to guide vascular cells to form sinusoid-like capillary structures.

In accordance with one embodiment, the inner surfaces of the macrochannels are lined with endothelial cells that originate from a sacrificial ink.

In accordance with one embodiment, the encapsulated cells include a co-culture of human mesenchymal stem cells and endothelial cells, and wherein the endothelial cells self-organize into a branched capillary plexus along the interstitial voids of the microporous matrix, guided by the topographical features of the matrix.

In accordance with one embodiment, the perfusable network of macrochannels comprises at least one inlet and at least one outlet port, configured to permit bidirectional perfusion through the tissue construct.

In accordance with one embodiment, the microporous matrix mimics liver sinusoidal microarchitecture.

In another aspect, the present invention provides a system for bioprinting vascularized tissue constructs. The system includes a programmable, multi-nozzle 3D bioprinter with at least two independently controlled printheads, one or more temperature-controlled cartridge dispensers for bioinks, a photocrosslinking apparatus configured to deliver blue light (405 nm), and a chamber for holding a self-healing support bath during embedded or suspension printing. The at least one of the temperature-controlled cartridge dispensers is configured to dispense a gelatin-based sacrificial bioink at 20-30° C.

In accordance with one embodiment, the system further includes a dual-syringe Y-connector for mixing a cell suspension into the matrix bioink under aseptic conditions.

In accordance with one embodiment, the system further includes nozzles with different gauge sizes for generating vascular structures with distinct channel diameters.

In accordance with one embodiment, the at least one controlled printhead is pneumatic-pressure driven and digitally controlled to regulate extrusion.

In accordance with one embodiment, the system further includes a microfibrous bioink preparation apparatus including a nylon mesh (e.g., a 20.0 μm nylon membrane filter) of approximately 20.0 μm pore size and a planetary centrifugal mixer.

In accordance with one embodiment, the system further includes a PDMS well chamber configured to contain the self-healing support bath during suspension printing.

Compared to conventional methods that rely solely on macrochannel printing or microvascular assembly, the present invention implements both strategies in a coordinated and reproducible manner. The platform supports modularity across tissue types, compatibility with various bioink compositions and cell sources, and scalability for larger constructs. Experimental results demonstrate enhanced nutrient diffusion, high cell viability, formation of endothelial-lined vessels, and preliminary in vivo integration. Applications include regenerative medicine, organ-specific disease models, and high-throughput drug testing using physiologically relevant vascularized tissues.

Key innovations of the present invention include the implementation of a dual-bioink strategy, multi-scale vascularization, and programmable multi-nozzle bioprinting. Specifically, the method employs a matrix bioink composed of gelatin, alginate, and gelatin methacrylate (GelMA), and a sacrificial bioink containing hyaluronic acid, gelatin, and calcium agents, which may be optionally loaded with endothelial cells. This dual system enables precise spatial control over cell distribution and the formation of both micro-capillary networks and perfusable macro-channels. The matrix bioink undergoes in-situ crosslinking via visible light and calcium ion exchange, enhancing construct integrity while preserving microporosity. The fibrous microgel matrix exhibits thixotropic and self-healing properties, supporting multiple printing modalities including interlaced, embedded, and suspension printing. Furthermore, the incorporation of a 1:1 co-culture system of human umbilical vein endothelial cells (HUVECs) and mesenchymal stem cells (hMSCs) enhances endothelial sprouting and lumen formation, while a time-delayed sacrificial ink based on 7.5% gelatin facilitates the gradual formation of large, endothelial-lined vascular channels. Preliminary in vivo results demonstrate promising integration potential, including host cell infiltration and human-specific vascular marker expression. Collectively, these features establish a versatile and scalable bioprinting platform capable of producing physiologically relevant, vascularized tissue constructs for applications in regenerative medicine, disease modeling, and drug discovery.

### BRIEF DESCRIPTION OF THE DRAWINGS

Embodiments of the invention are described in more details hereinafter with reference to the drawings, in which:

FIGS. 1A-1B illustrate a schematic representation of the multi-cellular bioprinting method for tissue constructs. (FIG. 1A) Illustration of direct ink writing (DIW)-based sacrificial bioprinting: Interlaced printing of degradable matrix and sacrificial bioinks. Post-incubation removal of sacrificial materials creates interconnected channels. (FIG. 1B) Multi-scale vascularization approach: Combination of microfibrous bioinks for capillary networks (using human mesenchymal stem cells (hMSCs) and human umbilical vein endothelial cells (HUVECs)) and sacrificial bioinks for larger vessels (using HUVECs).

FIG. 2A shows confocal micrographs comparing the nanoporous structure of a conventional bulk hydrogel with the interconnected microporous network of the fibrous microgel. Scale bars=200 μm. FIG. 2B shows rheological assessment demonstrating the thixotropic and self-healing properties of the fibrous microgel under alternating high (100%) and low (1%) strain conditions, in comparison with the stable, solid-like response exhibited by the conventional bulk hydrogel. FIG. 2C shows time-lapse images demonstrating the rapid permeation of 2000 kDa fluorescent dextran (blue) through the fibrous microgel, whereas the conventional bulk hydrogel remains impermeable, thereby illustrating the superior mass transport properties of the fibrous microgel.

FIGS. 3A-3C show degradation analysis of dual-bioink printed constructs. (FIG. 3A) Images of printed construct degradation over 10 days. Scale bar: 5 mm. (FIG. 3B) Remaining weight of printed constructs before and after immersion in PBS with varying calcium chloride concentrations. (FIG. 3C) 10-day degradation rates of constructs with varying calcium concentrations.

FIG. 4 depicts a graph showing the compression modulus of the fibrous microgel (MF) and the matrix bulk hydrogel (Bulk) following photocrosslinking with 405 nm light for various durations. The compression modulus of native mouse liver is included as a physiological benchmark for comparison.

FIGS. 5A-5B illustrate dual-bioink method for void channel formation in 3D bioprinted constructs. (FIG. 5A) Schematic of the in-situ crosslinking approach for bioprinting matrix and sacrificial inks. (FIG. 5B) Printing fidelity comparison: (i) calcium-free and (ii) 1% calcium printed lattice structures. Scale bar: 1 mm. (iii) Small-scale printed constructs. Scale bar: 5 mm. (iv) Void channel formation using a 25-gauge nozzle. Scale bar: 400 μm. (v-viii) Interlaced structures and void channel formation using a 30-gauge nozzle. Scale bar: (v&vii) 1 mm, (vi&viii) 400 μm.

FIG. 6A illustrates a conceptual schematic showing how multi-material bioprinting of matrix and sacrificial bioinks generates a porous tissue construct with a pre-designed, heterogeneous cellular organization after incubation. FIG. 6B shows schematics (top) and corresponding optical microscopy images (bottom) detailing the layer-by-layer fabrication process of the bioprinted construct. The workflow illustrates the orthogonal stacking of interlaced filaments composed of matrix and sacrificial bioinks, followed by the removal of the sacrificial material to yield a porous matrix scaffold.

FIG. 7A shows a macroscopic view of a printed hepatocyte-laden construct. FIG. 7B shows optical microscopy images illustrating the microstructure and cellular organization within the printed construct. FIG. 7C shows confocal microscopy images demonstrating the spatial patterning and interactions of multiple cell types within the bioprinted construct. FIG. 7D shows confocal images of a co-cultured construct containing human mesenchymal stem cells (hMSCs, green) and human umbilical vein endothelial cells (HUVECs, deep red). Scale bars=200 μm.

FIG. 8A illustrates a schematic illustration of the subcapsular liver implantation procedure used for in vivo evaluation of the bioprinted constructs. FIG. 8B shows a gross view of an explanted liver seven days post-implantation, with the bioprinted hydrogel construct visibly retained at the surgical site (outlined by a dashed line). FIG. 8C shows comparative hematoxylin and eosin (H&E) staining of liver tissues seven days post-implantation. (i) The acellular bulk hydrogel control exhibits a distinct boundary with the host tissue and lacks cellular infiltration. (ii) The cell-laden printed construct displays extensive host blood cell infiltration (indicated by black arrows) and seamless integration with the surrounding hepatic tissue. FIG. 8D shows immunohistochemical analysis of the cell-laden bioprinted construct. (i) Human CD31-positive endothelial structures are detected and indicated by black arrows. (ii) Human CD133-positive cells are similarly identified by black arrows. Scale bars=200 μm.

FIG. 9A illustrates a schematic representation of the native liver's stacked hexagonal lobule architecture, which serves as the biomimetic design inspiration for the vascularized tissue construct. FIG. 9B shows the embedded printing strategy, including (A) the microfibrous matrix bioink, (B) the sacrificial ink patterned into repeating lobule-like units, and (A+B) the process of embedding the sacrificial ink within the matrix to generate a biomimetic hexagonal architecture. FIG. 9C shows the final printed construct containing the embedded sacrificial network. The inset illustrates a magnified schematic of a single repeating unit after removal of the sacrificial material, revealing triangular tissue domains surrounded by interconnected perfusable channels.

FIG. 10A shows an illustration of the experimental setup for perfusion through the printed vascular channels within the bioprinted construct. FIG. 10B shows a schematic illustrating the concept of multi-scale vascularization achieved via suspension bioprinting, integrating large perfusable macrovascular channels with self-assembled microvascular networks formed within the fibrous matrix.

FIG. 11A shows a schematic illustration of the in-situ vascularization process, depicting the sequential progression from initial endothelial cell deposition along the channel walls to the formation of sprouting vascular structures that invade the surrounding matrix. FIG. 11B shows fluorescent images of HUVEC behavior within the bioprinted construct. (i) At day 1, initial cell deposition and aggregation along the channel walls are observed. (ii) By day 7, HUVECs exhibit significant proliferation and invasion into the surrounding matrix, forming elongated vascular sprouts. Scale bar=200 μm.

FIG. 12A shows the formation of an extensive, interconnected capillary network by day 10 within the fibrous microgel, generated by the co-culture of endothelial cells and mesenchymal stem cells. FIG. 12B shows the maturation of the capillary network by day 14, with the formation of distinct hollow lumens (indicated by asterisks), representing a key structural feature of functional microvasculature. Scale bar=50μm.

FIGS. 13A-13D show dual-bioink method for multi-cellular tissue constructs bioprinting. (FIG. 13A) Cell viability in matrix bioink: (i) Live/dead staining images at varying cell densities; (ii) Quantitative cell viability analysis. (FIG. 13B) Post printing after sacrificial ink removal (i-ii). Compact tissue-like structure formation after 48 hours (iii). Scale bar 200 μm. (FIG. 13C) Multi-cellular printing constructs: (i-iii) Interlaced printing pattern of matrix (M) and sacrificial (S1, S2) bioinks, scale bar 5 mm; (iv-viii) Cellular organization with different types in printed constructs. (FIG. 13D) Cells released from sacrificial ink adhere to matrix surface, forming void channels within printing constructs. Scale bar: 200 μm.

FIG. 14A shows an architectural comparison based on H&E staining of mouse liver tissue, revealing the native sinusoidal architecture. This structure is shown to be analogous to the network of interstitial voids formed by the spatially aligned fibers within the fibrous microgel. FIG. 14B shows a schematic illustrating the process of guided self-assembly, in which co-cultured HUVECs and hMSCs utilize the fibrous microgel architecture as a structural template to form an endothelialized network within the interstitial void spaces. FIG. 14C shows a confocal micrograph stained for F-actin and DAPI after 14 days of culture, revealing that the cells have self-organized into a continuous and interconnected network that mimics the structure of liver sinusoids, demonstrating the topographical guidance provided by the fibrous microgel.

FIGS. 15A-15D show morphological comparison of cells cultured in bulk and microfibrous hydrogels. (FIGS. 15A-15B) Cells encapsulated in bulk hydrogel. (FIGS. 15C-15D) Cells laden in the microfibrous hydrogel. Scale bar: 100 μm.

FIG. 16 shows time-course comparison of HUVEC mono-culture and HUVEC-hMSC co-culture in microfibrous hydrogel over 7 days. Scale bar: 50 μm.

FIG. 17 shows extended culture of HUVEC monoculture and HUVEC-hMSC co-culture in microfibrous hydrogel at day 10 and day 14. Lumens are marked with asterisks (*). Scale bar: 50 μm.

FIGS. 18A-18E show 3D bioprinting and development of macrovascular structures using HUVEC-laden sacrificial bioink. (FIGS. 18A-18B) 3D printed constructs demonstrating the scalability and reproducibility of the dual-bioink hydrogel platform. Scale bar: 1 cm. (FIG. 18C) HUVEC distribution within the sacrificial ink channel at day 1, showing initial cell aggregation. (FIG. 18D) HUVEC organization within and around the sacrificial ink channel at day 7, demonstrating cell invasion into the surrounding matrix. (FIG. 18E) F-actin staining revealing sprouted vascular structures and the void channel space, indicative of nascent blood vessel formation. Scale bar for E-G: 200 μm.

### DETAILED DESCRIPTION

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

“Matrix bioink” refers to a bioink formulation comprising at least one structural or cell-supporting hydrogel component, such as gelatin, gelatin methacrylate (GelMA), alginate, or combinations thereof, which forms the primary tissue matrix and provides a biologically permissive environment for cell encapsulation, migration, and tissue development. In preferred embodiments, the matrix bioink is photocrosslinkable and may include additional agents such as photoinitiators and ionic crosslinkers.

“Sacrificial bioink” refers to a printable hydrogel composition that is deposited to form a temporary structure within a bioprinted construct and is subsequently removed under physiological or aqueous conditions, such as thermal liquefaction or enzymatic degradation. In some embodiments, the sacrificial bioink may further comprise living cells, such as endothelial cells, which adhere to exposed surfaces after removal of the sacrificial material.

“Perfusable channels” or “macrochannels” refer to spatially defined voids or conduits formed within a bioprinted tissue construct, having dimensions suitable for convective fluid transport, and configured to allow flow of nutrients, oxygen, or blood substitutes. These channels are typically created by removing a sacrificial bioink and may be lined with endothelial cells to mimic natural vasculature.

“Microporous matrix” refers to a hydrogel-based structure comprising interconnected void spaces or pores with characteristic dimensions typically ranging from 5 to 100 μm. These micropores facilitate cell infiltration, nutrient diffusion, and self-assembly of microvascular networks, and may be formed by densely packed or jammed microgel particles.

“Fibrous microgel” or “fibrous microgel particles” refers to a suspension of crosslinked hydrogel fragments or strands produced by fragmenting a bulk hydrogel (e.g., via extrusion through a mesh), having diameters in the micron scale, and forming a jammed network upon packing. These fibrous elements provide a microporous architecture with enhanced rheological properties, including shear-thinning and self-healing behavior.

“Self-healing support bath” refers to a printable medium, such as a fibrous microgel matrix, that exhibits reversible thixotropic behavior—fluidizing under shear and rapidly re-solidifying upon removal of shear stress. This property enables the medium to support printed filaments during embedded or suspension bioprinting while maintaining structural integrity.

“Self-organized microvascular plexus” refers to a network of microvessels or capillary-like structures formed through spontaneous morphogenetic processes by embedded endothelial or vascular progenitor cells, typically guided by matrix topography, cell-cell interactions, and biochemical cues, without requiring pre-patterned channel formation.

“Hierarchical porosity” refers to the presence of interconnected pore structures at multiple size scales within a tissue construct, typically comprising larger perfusable macrochannels and smaller micropores or interstitial voids, which together facilitate both convective and diffusive mass transport.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

In the following description, method and system for multi-cellular bioprinting of vascularized tissue constructs are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The present invention provides a multi-cellular bioprinted tissue construct comprising an integrated, hierarchical vascular network, and a method for fabricating the same using a dual-bioink, multi-nozzle 3D bioprinting system. The invention overcomes limitations in conventional tissue engineering by enabling the scalable formation of both perfusable macrovascular channels and biologically self-organized microvascular networks within a single construct. The invention offers a platform-level solution for engineering thick, functional, and vascularized tissues suitable for regenerative medicine, disease modeling, and drug screening applications.

In one aspect, the present invention provides a method for fabricating a vascularized tissue construct through a multi-material, multi-cellular bioprinting process. The method integrates two complementary vascularization mechanisms: (i) top-down bioprinting of perfusable macrovascular channels and (ii) bottom-up self-organization of capillary-like microvessels. The fabrication process involves co-printing at least two distinct bioinks—a matrix bioink and a sacrificial bioink—into a spatially defined three-dimensional architecture using a programmable, multi-nozzle 3D bioprinter.

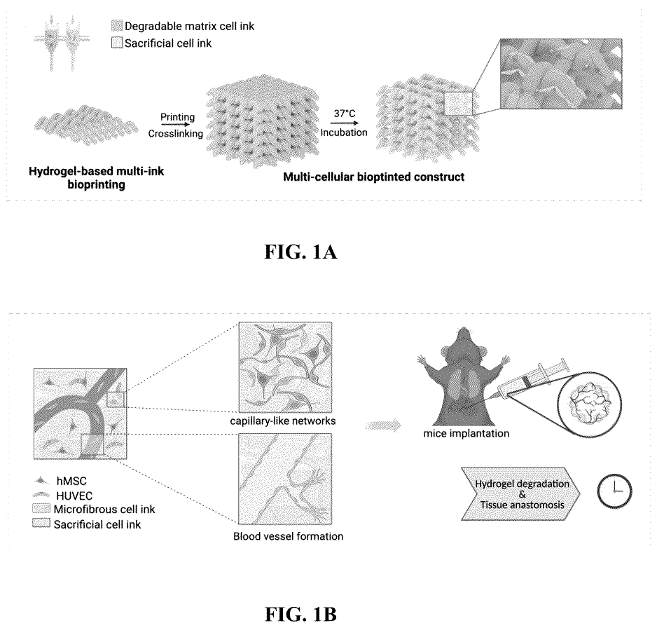

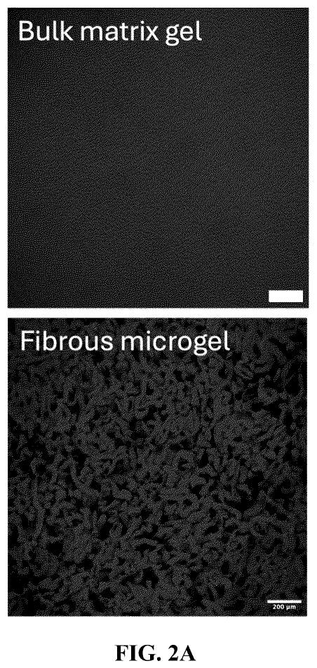

As shown in FIG. 1A, the method integrates two complementary mechanisms: (i) top-down bioprinting of perfusable macrovascular channels and (ii) bottom-up self-organization of capillary-like microvessels. The platform enables spatially controlled deposition of multiple bioinks using a programmable multi-nozzle bioprinter, wherein one bioink serves as a degradable matrix and the other functions as a sacrificial template. The degradable matrix bioink provides a microporous, biologically permissive environment that supports cell migration, proliferation, and morphogenesis, while the sacrificial bioink forms spatially defined conduits that can be selectively removed after printing to yield open channels. Together, these components allow the formation of multi-scale, continuous, and interconnected vascular structures within the construct.

In preferred embodiments, the matrix bioink comprises a fibrous microgel composed of gelatin, GelMA, alginate, and a suitable photoinitiator, forming a printable and crosslinkable hydrogel network. This fibrous matrix provides a microporous, biologically permissive environment that facilitates nutrient transport, cell migration, and vascular morphogenesis. The sacrificial bioink comprises at least one hydrogel-forming component, such as hyaluronic acid or gelatin, and may optionally encapsulate endothelial cells. Upon deposition, the matrix bioink is crosslinked via photopolymerization and/or ionic interactions, with calcium ions released from the sacrificial ink optionally promoting in situ crosslinking of the alginate component.

The sacrificial bioink is printed in a predefined pattern and subsequently removed under mild aqueous or physiological conditions, such as thermal liquefaction or enzymatic degradation, thereby generating perfusable macro-scale channels within the construct. Where the sacrificial ink is cell-laden, the released endothelial cells may adhere to the channel walls and promote in situ endothelialization. Concurrently, stromal and endothelial cells embedded in the matrix bioink undergo self-assembly to form an integrated microvascular plexus guided by the fibrous microarchitecture. This integrated fabrication strategy enables the formation of a multi-scale, continuous, and interconnected vascular system within the tissue construct. By modulating bioink compositions, cell types, and printing geometries, the method may be adapted for various tissue models, including organ-specific constructs for regenerative medicine, disease modeling, and drug discovery.

The invention further discloses three bioprinting modalities that leverage the properties of the fibrous microgel for scalable tissue engineering:
Interlaced co-printing: Alternating deposition of matrix and sacrificial inks creates interwoven channel networks, enabling patterned vascularization and spatially localized cell positioning. In vivo data confirm successful integration and enhanced cell survival compared to non-porous controls;
Embedded printing: By using the fibrous microgel as a cell-permissive matrix, complex vascular patterns (e.g., hepatic lobule analogs) can be printed directly within the construct, overcoming mass transport limitations of bulk gels; and
Suspension printing: The most advanced modality uses the fibrous microgel as a dynamic support bath. This enables simultaneous top-down printing of macro-vessels and bottom-up, matrix-guided self-assembly of capillary plexuses, generating a continuous, multi-scale vascular tree that mimics native organ architecture.

In another aspect, the present invention provides an integrated, multi-material bioprinting platform and a bioprinted tissue construct comprising an integrated, hierarchical vascular network within a biocompatible, degradable matrix. The platform utilizes a programmable, multi-nozzle 3D bioprinter and a dual-bioink strategy to enable spatially defined deposition of both matrix and sacrificial materials. A key innovation of the system is the use of a fibrous microgel matrix bioink, which exhibits a microporous architecture with interconnected voids that facilitate nutrient diffusion, cell infiltration, and capillary self-assembly. This fibrous microgel not only supports high cell viability and mass transport, but also provides topographical cues that guide embedded cells to self-organize into capillary-like structures, thereby forming a biologically relevant microvascular plexus.

Simultaneously, the sacrificial bioink is printed in a predefined pattern and subsequently removed to generate macro-scale perfusable channels within the construct. These hollow conduits may be endothelialized to mimic native vasculature, resulting in a dual-scale vascular network that closely replicates the structural and functional complexity of native tissues. In preferred embodiments, the matrix bioink is co-loaded with a population of endothelial cells, such as HUVECs, and hMSCs, which synergistically enhance vascular sprouting, endothelial alignment, and lumen formation throughout the construct.

FIG. 1B depicts an example of a multi-cellular bioprinted tissue construct comprising a hierarchical vascular network. Perfusable macrochannels are embedded within the printed matrix and can be seeded with endothelial cells, while interstitial cells co-encapsulated within the matrix undergo self-assembly to form microvessels. This integrated vascular system enhances nutrient diffusion and oxygen transport across the construct and enables the fabrication of thick, viable, and physiologically relevant engineered tissues.

The fibrous microgel exhibits a microporous network of interconnected voids, mimicking native liver sinusoidal topography and facilitating biological self-assembly. Its tunable mechanical properties and self-healing rheology allow it to function both as a tissue matrix and a support bath for complex embedded or suspension printing. In preferred embodiments, this bioink enables capillary network formation by encapsulated endothelial and stromal cells without the need for pre-patterned microchannels.

The method and system of the present invention demonstrate potential for creating a wide range of vascularized tissue constructs, with possible applications in tissue engineering and regenerative medicine. Further development may involve optimization of printing parameters, exploration of additional cell types and biomaterials, and investigation of scalability for potential clinical applications.

### EXAMPLE

### Example 1—Materials and Methods

### Multi-Nozzle Bioprinting Process

In one embodiment, a multi-nozzle direct ink writing (DIW) bioprinting process was employed for the fabrication of multi-material and/or multi-cellular constructs. The system utilized was a GeSiM DIW bioprinter equipped with three independent pneumatic pressure-based dispensing modules, each digitally controlled to enable programmable deposition of distinct bioinks. Each bioink formulation was loaded into individual sterile cartridge dispensers coupled to nozzles of different inner diameters, selected based on the rheological characteristics of the corresponding ink. Prior to bioprinting, the temperature control platform and dispensing modules were pre-set to the desired operating temperature (typically between 15° C. and 25° C.), and maintained under stable conditions for a minimum of one hour to ensure thermal equilibrium.

Printing parameters were systematically optimized to ensure continuous and uniform filament extrusion. In particular, the printing velocity was controlled within a range of 6 to 8 mm/s, and the pneumatic extrusion pressure was adjusted between 30 to 100 kPa, depending on the specific viscosity and shear-thinning behavior of each bioink. In addition to pressure calibration, the volumetric flow rate was monitored and maintained between 0.05 and 0.2 mL/min, depending on the nozzle diameter and material properties, to ensure dimensional fidelity and reproducible deposition.

Immediately following the deposition process, the printed constructs were subjected to photocrosslinking by exposure to visible blue light with a wavelength of 405 nm and an intensity of 25 mW/cm² for a duration of 60 seconds, sufficient to induce rapid network polymerization of the photo-reactive GelMA components. In certain embodiments, an optional ionic crosslinking step was performed by immersing the crosslinked constructs in a sterile aqueous calcium chloride (CaCl₂) solution at concentrations ranging from 0 to 1 wt % for 1 to 3 minutes, thereby promoting additional ionic crosslinking of alginate components within the matrix. Afterward, the constructs were thoroughly rinsed with phosphate-buffered saline (PBS).

The resulting constructs were then immersed in Dulbecco's Modified Eagle Medium (DMEM) and transferred to a humidified cell culture incubator maintained at 37° C. and 5% CO₂ for post-fabrication incubation and cellular recovery. All procedures, including bioink preparation, system calibration, bioprinting, photocrosslinking, and post-processing, were conducted under aseptic conditions to ensure sterility throughout the fabrication workflow.

### Cell-Laden Bioink Preparation

In one embodiment, a cell-laden matrix hydrogel bioink was prepared under aseptic conditions. Specifically, 1 mL of the sterilized matrix hydrogel ink was drawn into a sterile disposable syringe. Separately, a concentrated cell suspension was prepared by resuspending the desired cell population in a sterile medium supplemented with fibronectin at a final concentration of approximately 1 mg/mL, yielding a cell density in the range of 1×10⁶ to 1×10⁷ cells/mL.

The hydrogel ink syringe and the cell suspension syringe were connected via a sterile dual-lumen mixing interface, such as a Y-connector or a two-way valve. The contents of the two syringes were mixed by gently and alternately actuating the plungers in a push-pull motion, thereby ensuring uniform cell distribution throughout the hydrogel matrix while minimizing air bubble formation and mechanical shear on the cells.

Following mixing, the resulting homogeneous cell-laden hydrogel ink was transferred to a sterile dispensing cartridge or syringe pre-fitted with a suitable printing needle (e.g., inner diameter selected according to the bioink viscosity and cell size). The formulation was immediately ready for use in subsequent bioprinting processes involving DIW or extrusion-based deposition.

### Cell Staining and Imaging

Cell staining was performed using a membrane-permeable fluorescent dye to enable subsequent visualization and tracking of cells within printed constructs. Specifically, CellTracker™ Blue or CellTracker™ Deep Red dye (Invitrogen™, Thermo Fisher Scientific) was used for pre-labeling cells prior to bioprinting.

The dye was diluted to a final working concentration of 1 to 10 μM in DMEM without serum. The resulting staining solution was added to adherent or suspended cell cultures and incubated at 37° C. for 30 minutes under standard culture conditions. Following incubation, cells were washed three times with fresh medium or PBS to remove excess dye. The stained cells were then collected by centrifugation, resuspended in sterile buffer or bioink-compatible solution, and subsequently used for encapsulation in bioinks or direct printing.

### Immunofluorescence Staining

Immunofluorescence staining was performed to visualize cytoskeletal structures and endothelial markers within the bioprinted constructs. All procedures were conducted under sterile conditions unless otherwise specified.

The samples were first washed with PBS and then fixed in 3.7% paraformaldehyde for 10 minutes at room temperature. Following fixation, the samples were permeabilized using a standard permeabilization solution (e.g., PBS containing 0.1% Triton X-100).

For F-actin staining, a fluorescent phalloidin conjugate (e.g., F-actin-Green-488) was diluted at 1:40 in PBS and applied to the samples for 40 minutes in the dark at room temperature. After incubation, the samples were washed thoroughly using PBS containing 0.1% Triton X-100 to remove unbound dye. The samples were then counterstained with 4′,6-diamidino-2-phenylindole (DAPI) at a concentration of 10 μg/mL for nuclear visualization, followed by additional PBS washes.

For CD31 immunostaining, the fixed and permeabilized samples were first blocked with 3% bovine serum albumin (BSA) in PBS for 1 hour at room temperature to minimize non-specific antibody binding. The samples were then incubated overnight at 4° C. with a primary anti-CD31 antibody (e.g., 1:100 dilution in blocking buffer). After incubation, samples were washed thoroughly using PBS containing 0.1% Triton X-100. A secondary antibody conjugated with a fluorescent dye (e.g., Alexa Fluor™ 594 anti-mouse or anti-rabbit IgG) was then applied at a dilution of 1:500 and incubated for 1 hour at room temperature in the dark. A final DAPI counterstaining (10 μg/mL) was performed as described above, followed by multiple PBS washes to remove excess stain.

The stained samples were then mounted on glass slides or maintained in imaging-compatible culture dishes for subsequent fluorescence or confocal microscopy, allowing visualization of cytoskeletal organization and endothelial marker expression within the 3D bioprinted constructs.

### In Vivo Transplantations and Analysis

In vivo transplantation studies were conducted to evaluate the biocompatibility and integration of bioprinted hydrogel constructs in a mammalian model. Cylindrical constructs with a 5 mm inner diameter and 2 mm height were fabricated using the previously described cell-laden matrix bioinks. Prior to transplantation, the constructs were cultured in vitro for 3 days under standard conditions (37° C., 5% CO₂) to promote early-stage microvascular network formation.

As a control, acellular hydrogel constructs of identical geometry and composition were fabricated and cultured under identical conditions.

Transplantation procedures were performed using 10-week-old athymic nude (Nu/Nu) immunocompromised mice (n=3). All animal handling and surgical procedures were conducted in accordance with institutional animal care and use guidelines. Mice were anesthetized using inhaled isoflurane, and a small surgical incision was made to expose the left lateral hepatic lobe. PBS was injected under the hepatic capsule to gently separate the membrane from the underlying parenchyma, creating a subcapsular pocket for graft placement.

The bioprinted constructs were then carefully inserted into the created subcapsular space. The incision site was sutured closed, and mice were returned to standard housing with routine monitoring.

At 7 days post-transplantation, the animals were euthanized, and the liver lobes containing the implanted constructs were excised. The tissue samples were fixed in formalin, embedded in paraffin, and processed for hematoxylin and eosin (H&E) staining. Histological sections were examined for morphological assessment, including evaluation of hydrogel integrity, host tissue response, and preliminary evidence of integration between the construct and hepatic tissue at the subcapsular interface.

### Histological Analysis

Histological analysis was performed to assess the structural integrity and host tissue interaction of explanted hydrogel constructs following in vivo transplantation. The retrieved samples were first fixed in 10% neutral-buffered formalin for 2 hours at room temperature, followed by dehydration through a graded ethanol series and subsequent embedding in paraffin using standard histological techniques.

Paraffin-embedded samples were sectioned into 10 μm-thick slices using a rotary microtome. The sections were then mounted on glass microscope slides and subjected to standard deparaffinization and rehydration procedures, including sequential immersion in xylene, descending concentrations of ethanol (100%, 95%, 70%), and distilled water.

For H&E staining, the rehydrated tissue sections were incubated with hematoxylin for 3 to 5 minutes, rinsed in running tap water, and, if required, differentiated in 1% acid alcohol to reduce background staining. The sections were then blued in tap water or alkaline solution, followed by counterstaining with eosin for 1 to 2 minutes. After staining, the slides were sequentially dehydrated in graded ethanol, cleared in xylene, and mounted with a permanent coverslip.

All histological procedures were performed using standardized protocols to ensure reproducibility. The stained sections were subsequently examined by brightfield microscopy for analysis of tissue morphology, construct retention, inflammatory response, and integration at the graft-host interface.

### Immunohistochemistry Staining

Immunohistochemistry (IHC) staining was performed on histological sections to detect and localize specific cell types within the explanted bioprinted constructs. This analysis aimed to confirm the presence and spatial distribution of hMSCs and HUVECs within the transplanted tissues.

Paraffin-embedded tissue sections were prepared and subjected to standard deparaffinization, rehydration, and antigen retrieval procedures. Following blocking of endogenous peroxidase activity and non-specific binding, the sections were incubated with primary antibodies specific for target markers: a CD133 antibody for the identification of hMSCs, and a CD31 antibody for detection of HUVECs.

After incubation with the primary antibody, the sections were treated with a horseradish peroxidase (HRP)-conjugated secondary antibody, according to the manufacturer's recommended protocol. Signal development was achieved using 3,3′-diaminobenzidine (DAB) tetrahydrochloride as the chromogenic substrate. Colorimetric development was performed for approximately 3 minutes, allowing visible brown staining at the sites of antigen expression.

All steps were conducted in accordance with the antibody manufacturer's instructions. Stained slides were counterstained with hematoxylin where appropriate and examined under brightfield microscopy to assess cell localization, persistence, and potential tissue integration.

### Example 2—Bioinks Preparation

In this example, three types of bioinks were prepared for use in the fabrication of multi-scale vascularized tissue constructs, including: (1) a matrix hydrogel bioink, (2) a fibrous microgel bioink, and (3) a sacrificial bioink.

### Matrix Hydrogel Bioink

A degradable matrix hydrogel bioink was prepared by dissolving 2.5 wt % GelMA, 1-5 wt % gelatin, and 0.2 wt % lithium phenyl- 2,4,6-trimethylbenzoylphosphinate (LAP) in PBS. The components were heated to 50° C. until fully dissolved and subsequently sterilized using a 0.22 μm membrane filter. Sterile alginate powder (0-2 wt %) was then added to the solution under aseptic conditions. The resulting mixture was homogenized at 2500 rpm for 5 minutes using a planetary centrifugal mixer to ensure uniform distribution.

### Fibrous Microgel Bioink

To prepare the fibrous microgel bioink, a pre-gel solution was formulated containing 0-0.2 g alginate, 0.25 g gelatin, 0.25 g GelMA, and 0.02 g LAP dissolved in 10 mL PBS. The solution was sterilized, transferred into a dispensing cartridge, and refrigerated for approximately 1 hour to induce partial gelation. Photocrosslinking was then performed using blue light at an intensity of 25 mW/cm² for 20 seconds to stabilize the network. The crosslinked hydrogel was extruded through a 20 μm nylon mesh, producing a fibrous microgel with interconnected microporosity. When cell encapsulation was required, the fibrous microgel was mixed with a concentrated cell suspension using a dual-syringe connector to achieve homogeneous distribution.

### Sacrificial Bioink

The sacrificial ink was composed of 5-7.5 wt % gelatin dissolved in deionized water containing 0-1.0 wt % calcium chloride. The solution was maintained at 24-27° C. to retain printability. In certain embodiments, the sacrificial bioink was cell-laden. For this purpose, HUVECs were suspended at a concentration of approximately 1×10⁶ cells/mL and mixed with the sacrificial ink immediately prior to printing.

These bioinks were used in subsequent examples to fabricate vascularized constructs exhibiting both perfusable macrochannels and microcapillary networks.

### Example 3—Characterization of the Fibrous Microgel and Bioinks

This example describes the structural, rheological, and degradation-related characterization of the fibrous microgel matrix bioink used in the fabrication of hierarchical vascularized tissue constructs.

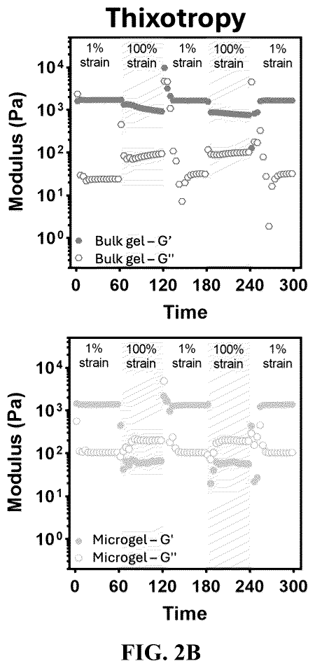

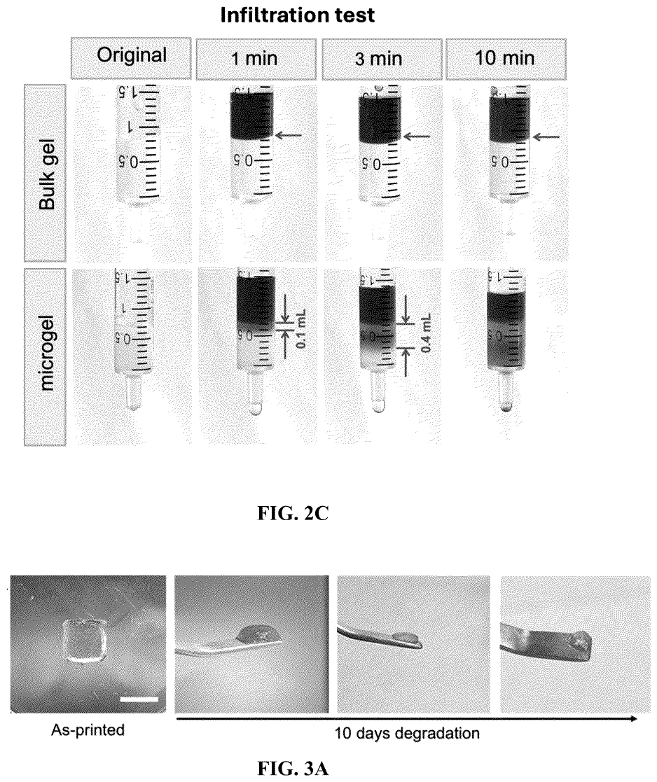

To overcome the limited mass transport associated with conventional bulk hydrogels, a fibrous microgel matrix was developed. Unlike solid bulk hydrogels, which possess a nanoporous network, the fibrous matrix is composed of discrete microgel particles forming a jammed, interconnected microporous architecture. As shown in FIG. 2A, confocal micrographs confirm the presence of micron-scale voids distributed throughout the construct. These interconnected micropores significantly increase available surface area and void space, enabling enhanced permeability and cell infiltration, which are key features for engineering thick, metabolically active tissues.

The fibrous microgel also exhibits distinct thixotropic behavior, characterized by shear-thinning and self-healing properties. Rheological measurements were performed using a five-step alternating strain protocol. As shown in FIG. 2B, the fibrous microgel undergoes reversible transitions between solid-like (G′>G″) and fluid-like (G″>G′) states under alternating low (1%) and high (100%) strain conditions. The storage modulus (G′) dropped below the loss modulus (G″) during high strain, indicating fluidization, and fully recovered upon return to low strain, indicating rapid self-healing. In contrast, conventional bulk gels retained a solid-like response throughout. This self-healing ability enables the fibrous matrix to function as a support bath during embedded and suspension bioprinting of complex structures.

To assess mass transport capabilities, diffusion assays using 2000 kDa fluorescent dextran were conducted. As illustrated in FIG. 2C, the dye rapidly permeated through the fibrous microgel matrix, indicating high permeability. Conversely, in bulk hydrogels, the dye remained confined to the surface, with no observable penetration. These results confirm that the microporous structure of the fibrous microgel significantly enhances solute transport, supporting improved nutrient diffusion and waste removal in large tissue constructs.

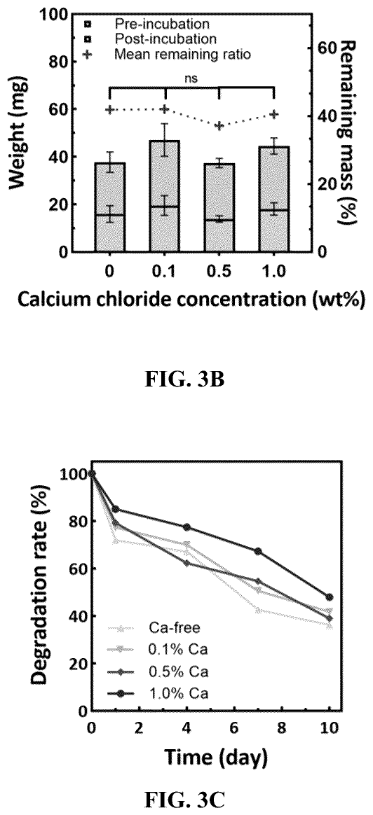

Post-printing stability and degradation of the constructs were also characterized. As shown in FIG. 3A, the sacrificial bioink was removed via immersion in PBS, leaving behind perfusable channels within the matrix. Quantitative weight analysis (FIG. 3B) indicated that approximately 50% of the initial construct mass remained after sacrificial ink removal. Over a 10-day incubation period, constructs containing higher calcium ion concentrations demonstrated increased structural integrity and slower degradation, as illustrated in FIG. 3C. These properties allow for gradual matrix turnover and potential replacement by native extracellular matrix components during tissue maturation.

### Example 4—Tunability of Bioink Mechanical Properties via Controlled Photocrosslinking

This example illustrates the ability to modulate the mechanical stiffness of engineered hydrogel constructs formed from the disclosed bioinks, thereby enabling the constructs to mimic the compression modulus of native soft tissues such as liver.

This example demonstrates that the mechanical properties of hydrogel constructs formed from the disclosed bioinks are tunable to mimic the stiffness of target native tissues. Constructs were fabricated from (i) a fibrous microgel matrix bioink (MF) and (ii) a matrix bulk hydrogel (Bulk) comprising the same base components but lacking the fibrous microarchitecture. Following printing, photocrosslinking was performed by exposing the constructs to 405 nm blue light at 25 mW/cm² for 30, 60, 120, or 240 seconds.

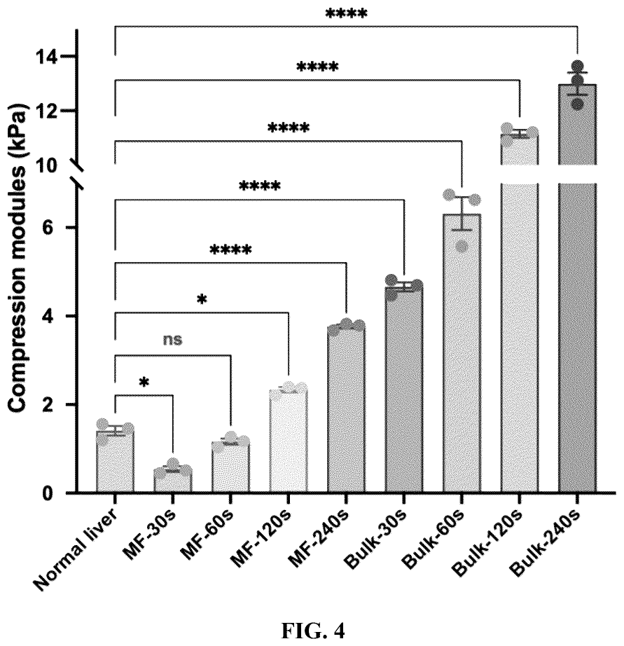

Unconfined compression testing was conducted on the crosslinked constructs to determine the compression modulus. As a physiological benchmark, native mouse liver samples were tested under identical conditions. The compiled results are presented in FIG. 4.

As shown in FIG. 4, both MF and Bulk constructs exhibited a dose-dependent increase in compression modulus with increasing light exposure time. At matched exposure durations, the Bulk constructs consistently displayed higher modulus than the MF constructs, reflecting the microstructural differences between bulk and fibrous matrices. Importantly, in one embodiment, the MF construct crosslinked for approximately 60 seconds yielded a compression modulus that was not significantly different from native mouse liver (indicated as ns in FIG. 2), whereas longer exposures (e.g., 120-240 seconds) produced progressively stiffer materials. Bulk constructs showed a similar monotonic trend, reaching kPa-level moduli exceeding those of MF at all exposure times.

These findings confirm that light-exposure duration provides a practical control knob for precision tuning of construct mechanics. This tunability enables the disclosed platform to match tissue-specific stiffness (e.g., liver-like for MF-60s) or to intentionally deviate to stiffer regimes when required for other targets, thereby broadening applicability across regenerative medicine, disease modeling, and drug screening.

### Example 5—Dual-Bioink Fabrication of Multi-Scale Vascularized Constructs

This section details three distinct but related printing architectures (prototypes) that leverage the disclosed bioinks to create progressively more complex and functional vascularized tissues.

### Example 5A

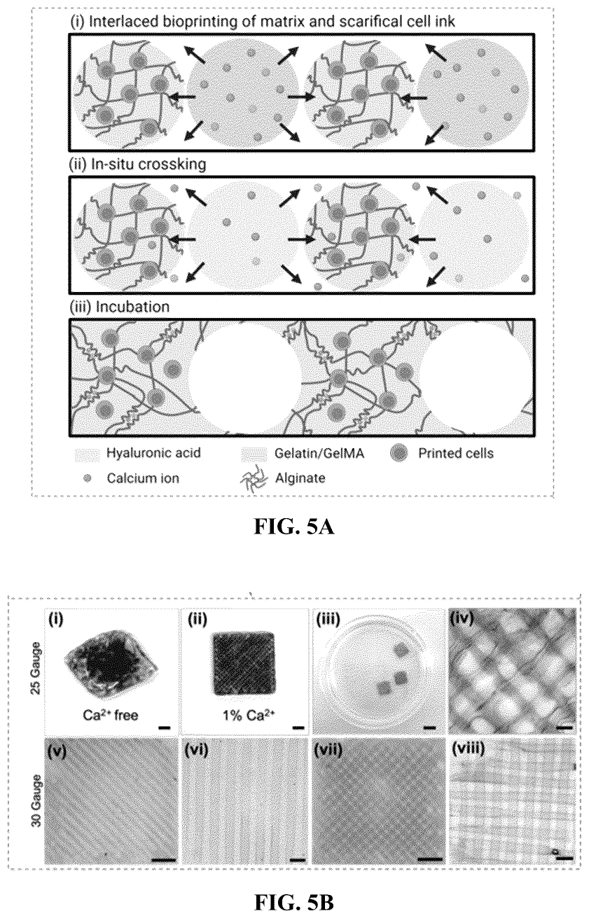

This example demonstrates the fabrication of vascularized tissue constructs using a dual-bioink printing method, wherein a structural matrix bioink and a removable sacrificial bioink were co-printed in an interlaced pattern to generate perfusable channels within a microporous support matrix. The fabrication process corresponds to the schematic steps illustrated in FIG. 5A and the experimental validation shown in FIG. 5B.

As shown in FIG. 5A(i), a multi-nozzle bioprinter was employed to deposit alternating filaments of the matrix bioink and the sacrificial bioink on a substrate. The matrix bioink comprised a blend of GelMA, gelatin, and optionally alginate, forming a microporous and biocompatible support phase. The sacrificial bioink was composed of hyaluronic acid and calcium ions, and in certain embodiments included embedded HUVECs. Immediately after printing, the construct underwent in situ crosslinking, as illustrated in FIG. 5A(ii). Photocrosslinking of the GelMA component was performed using blue light irradiation (25 mW/cm²) for 30 seconds. Concurrently, calcium ions originating from the sacrificial ink diffused into the adjacent matrix bioink, promoting ionic crosslinking of the alginate chains. This dual crosslinking strategy stabilized the construct while preserving interconnected microporosity. As shown in FIG. 5A(iii), the construct was then incubated at 37° C. under aqueous conditions to dissolve the hyaluronic acid-based sacrificial phase. The removal of sacrificial ink resulted in the formation of open, perfusable macrochannels within the crosslinked matrix. In cell-laden conditions, endothelial cells originating from the sacrificial ink adhered to the internal surface of the formed channels, enabling in situ endothelialization.

The performance of this method was further validated experimentally, as shown in FIG. 5B. Constructs printed without calcium (FIG. 5B(i)) showed structural collapse, while those printed with 1 wt % Ca²⁺ (FIG. 5B(ii)) formed well-defined lattice structures. Multiple grid and line patterns were fabricated using nozzles of 250 μm (25 G, FIG. 5B(iv)) and 150 μm (30 G, FIG. 5B(v-viii)) to evaluate resolution and material fidelity. The resulting constructs displayed high pattern fidelity and stable crosslinked geometry suitable for subsequent perfusion and cell culture.

Together, this dual-bioink approach enabled the fabrication of vascularized constructs comprising both engineered perfusable macrochannels and a microporous, cell-permissive matrix. This strategy facilitates spatially controlled vascular patterning and provides a robust foundation for hierarchical tissue engineering.

### Example 5B

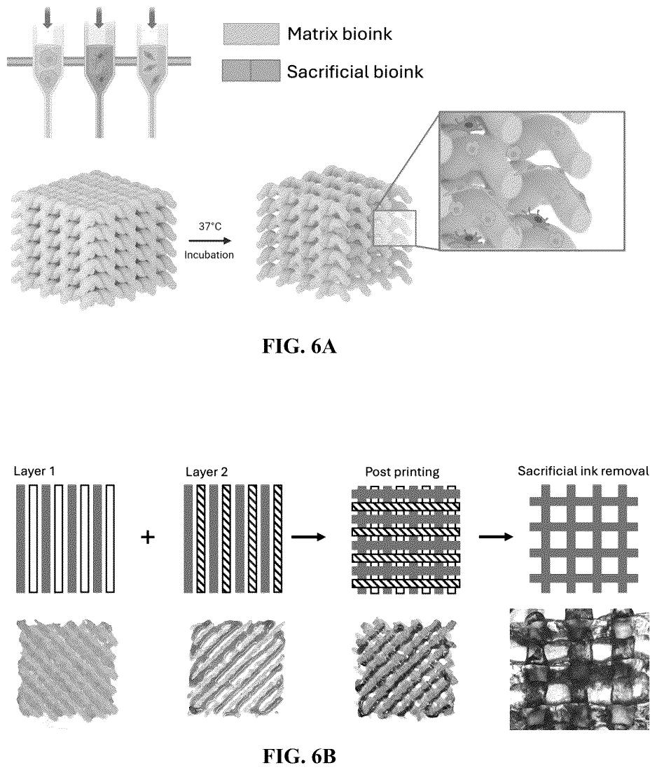

In one embodiment, a three-dimensional vascularized tissue construct was fabricated using an interlaced co-printing strategy that enables the spatially defined deposition of multiple cell populations and sacrificial elements. As schematically illustrated in FIG. 6A, a multi-nozzle direct ink writing system was employed to co-deposit at least one matrix bioink and one sacrificial bioink in an alternating filament arrangement. The matrix bioink provided long-term structural support and cellular encapsulation, while the sacrificial ink functioned as a transient placeholder for subsequent macrochannel formation.

As shown in FIG. 6B, orthogonally aligned filaments of the matrix and sacrificial inks were sequentially deposited in a layer-by-layer manner to build a lattice-like 3D architecture. Upon completion of printing, the matrix was crosslinked using photoirradiation, and the sacrificial component was subsequently removed by thermal or enzymatic means to generate interconnected perfusable macrochannels. This process resulted in the formation of a porous construct with internal conduits suitable for fluid transport and cell-cell communication.

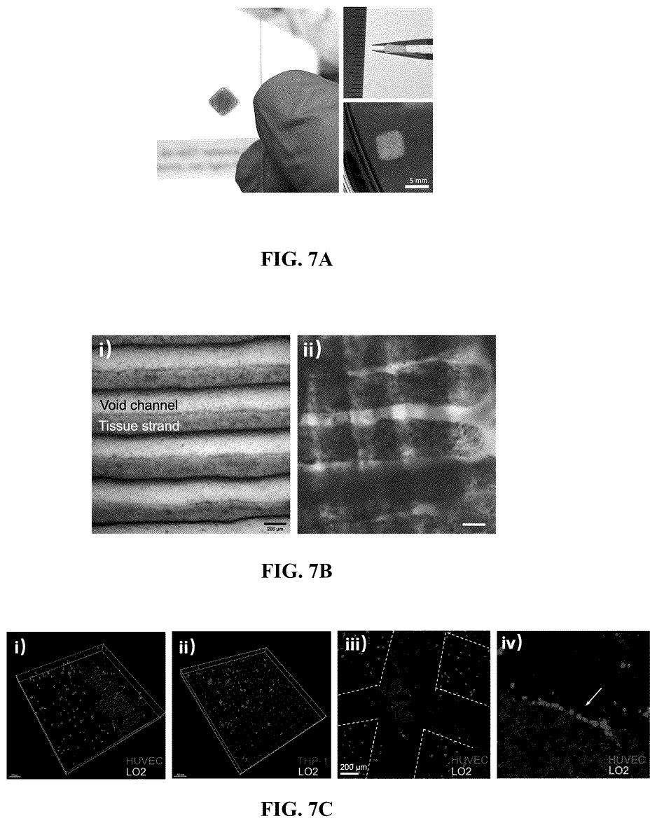

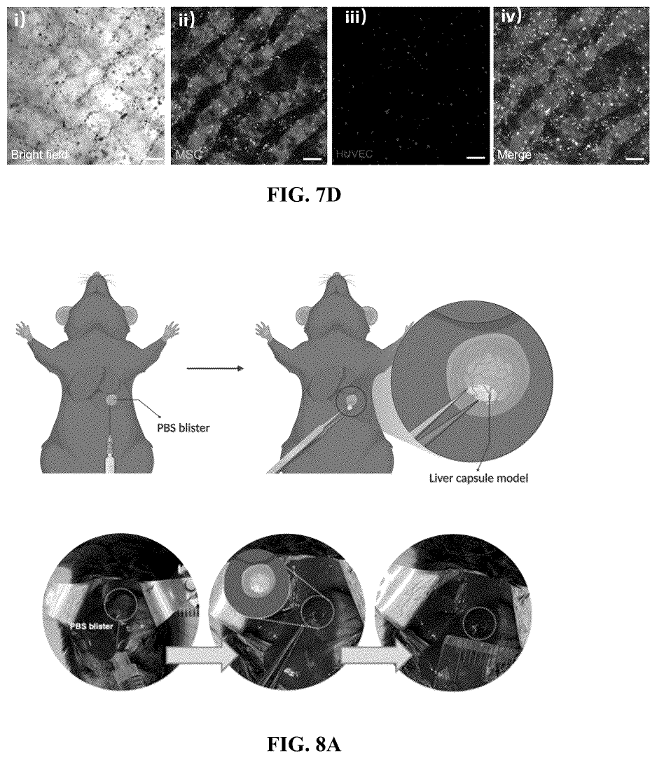

To evaluate the functionality of this architecture in supporting tissue formation, constructs were fabricated using a matrix bioink containing primary hepatocytes at a density of approximately 1×10⁷ cells/mL, interlaced with an acellular sacrificial ink. As illustrated in FIG. 7A, the resulting construct exhibited robust structural integrity. After 48 hours of incubation, compaction of hepatocyte-laden matrix strands was observed, forming dense cord-like tissue regions consistent with early-stage tissue morphogenesis (FIG. 7B).

In another embodiment, a tri-ink co-printing configuration was utilized to deposit three distinct bioinks, respectively containing fluorescently labeled human hepatocytes (LO2), endothelial cells (HUVECs), and immune cells (THP-1). As shown in FIG. 7C(i)-(iii), the printed cell filaments maintained clear spatial segregation within the lattice architecture. Importantly, after 24 hours of incubation, the HUVECs embedded within the sacrificial ink were observed to adhere to the interior surfaces of adjacent matrix filaments, forming endothelial-like linings along the macrochannel walls (FIG. 7C(iv)). This phenomenon represents in situ endothelialization induced by programmed release and adhesion.

Further experiments involving co-printing of hMSCs within the matrix ink and HUVECs within the sacrificial ink demonstrated the ability to engineer tissue constructs with regionally co-localized cell populations. As evidenced by confocal imaging in FIG. 7D, hMSCs remained confined within the matrix scaffold, while HUVECs migrated and adhered to the matrix interface, establishing spatial proximity conducive to vasculogenic signaling and lumen formation.

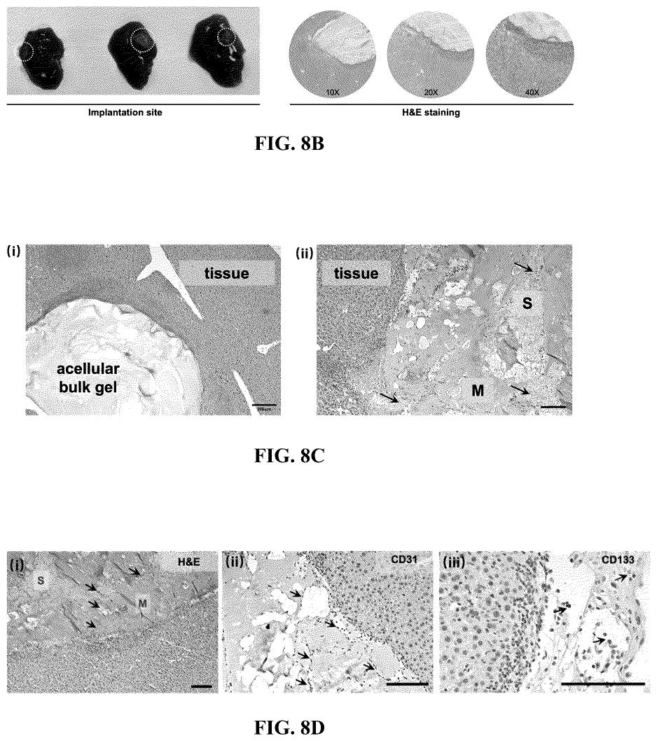

To assess the biointegration potential of the engineered constructs, cell-laden interlaced constructs were transplanted into the subcapsular hepatic region of immunocompromised murine hosts. Constructs fabricated using bulk acellular hydrogel served as controls. As illustrated in FIGS. 8A-8B, the printed constructs retained their anatomical positioning post-implantation. Hematoxylin and eosin (H&E) staining of explanted tissues revealed distinct differences in host response. In the control group, the hydrogel interface remained non-integrated and acellular (FIG. 8C(i)). In contrast, the printed constructs exhibited widespread host cell infiltration and a continuous interface with adjacent liver tissue (FIG. 8C(ii)).

To confirm human cell persistence and phenotype, immunohistochemical staining was performed on explanted tissue samples. Human endothelial marker CD31 was detected along lumen-like structures within the construct (FIG. 8D(i)), while mesenchymal stem cell marker CD133 was detected in the matrix regions (FIG. 8D(ii)), confirming in vivo survival and spatial organization of the implanted hMSCs and HUVECs.

These results collectively demonstrate that the interlaced co-printing architecture disclosed herein supports: (i) precise deposition of multiple bioinks and cell types, (ii) programmable formation of perfusable channels, (iii) in situ endothelialization without secondary seeding, and (iv) successful in vivo integration and persistence of human-derived tissue components. This strategy enables the fabrication of hierarchical, functionally organized tissue constructs with potential applications in regenerative medicine, disease modeling, and vascularized tissue replacement.

These in vivo results underscore the biological relevance of the interlaced co-printing architecture. The porous construct permitted substantial host blood cell infiltration, and the presence of human CD31⁺ and CD133⁺ markers confirms successful engraftment and survival of both endothelial and mesenchymal stromal cell populations. This supports the utility of the disclosed architecture for future clinical translation and long-term regenerative applications.

### Example 5C

In one embodiment, a vascularized tissue construct was fabricated via an embedded bioprinting architecture, wherein a sacrificial bioink was directly deposited into a fibrous microgel (MF) matrix that simultaneously served as a biofunctional tissue scaffold and as a shear-thinning support medium. This approach is configured to overcome limitations associated with conventional bulk hydrogels, particularly with respect to their nanoporous structure and restricted mass transport properties.

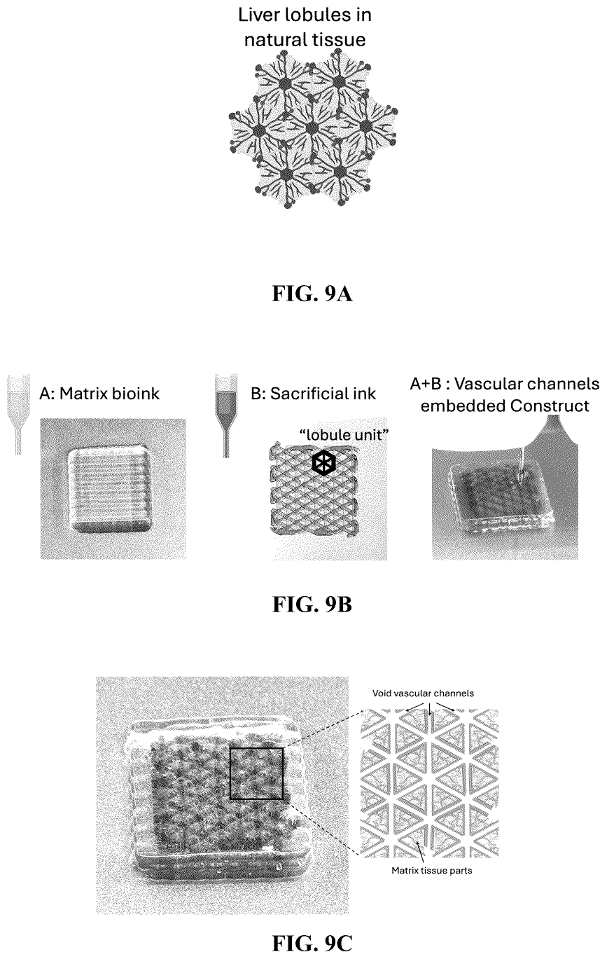

As schematically illustrated in FIG. 9A, the embedded printing architecture was inspired by the hierarchical vascular design of hepatic lobules, which consist of radially organized sinusoidal networks converging toward a central vein. To recapitulate this hexagonal microanatomy, a three-layered lattice printing scheme was implemented, as detailed in FIG. 9B.

The process comprised the following steps:
(i) Formation of support matrix: The fibrous microgel bioink (Bioink A), previously crosslinkable and microporous, was dispensed into a printing reservoir or mold to define the receiving tissue matrix and support bath;
(ii) Embedded sacrificial patterning: A sacrificial bioink (Bioink B), optionally cell-laden, was extruded into the MF matrix to form vascular templates. The printing followed a rotational pattern, wherein each of the three successive layers was deposited with a 60-degree angular offset, thereby generating repeating hexagonal geometries;
(iii) Crosslinking and stabilization: Upon completion of sacrificial ink deposition, the entire construct was stabilized via photo-crosslinking and/or ionic gelation, depending on the specific functional groups of the MF matrix; and
(iv) Removal of sacrificial component: The sacrificial ink was selectively removed under aqueous or thermal conditions to yield a perfusable macrochannel network embedded within the MF-based tissue matrix.

As shown in FIG. 9C, the resulting construct comprises a hierarchical, multi-scale porous architecture, consisting of: (i) engineered macro-scale vascular conduits, formed by the sacrificial ink removal, which define triangular tissue zones arranged in a biomimetic lobular configuration; and (ii) material-intrinsic micro-scale interstitial porosity, provided by the fibrous microgel matrix itself, enabling enhanced nutrient diffusion and cellular remodeling within each lobular domain.

Notably, each triangular tissue zone is defined by converging perfusable channels and is fully composed of the MF material, thereby providing continuous, interconnected microporosity throughout the entire construct. This structural feature affords a dual transport advantage: macro-scale perfusion through vascular conduits, and localized mass transfer within tissue domains.

Compared to the interlaced co-printing strategy described in Example 5B, the present architecture enables greater architectural precision, improved perfusion uniformity, and closer anatomical mimicry of tissues with lobular or nodular morphologies. In particular, the angular control over sacrificial lattice orientation permits the generation of isotropic or anisotropic vascular territories, allowing application to a wide range of organ systems including, but not limited to, liver, kidney, and pancreas.

### Example 5D

In one embodiment, a multi-scale vascularized tissue construct was fabricated via a suspension bioprinting architecture, wherein a sacrificial bioink was deposited within a fibrous microgel (MF) support bath that simultaneously functions as a (i) self-healing, shear-thinning deposition medium and (ii) biologically permissive, microporous tissue matrix.

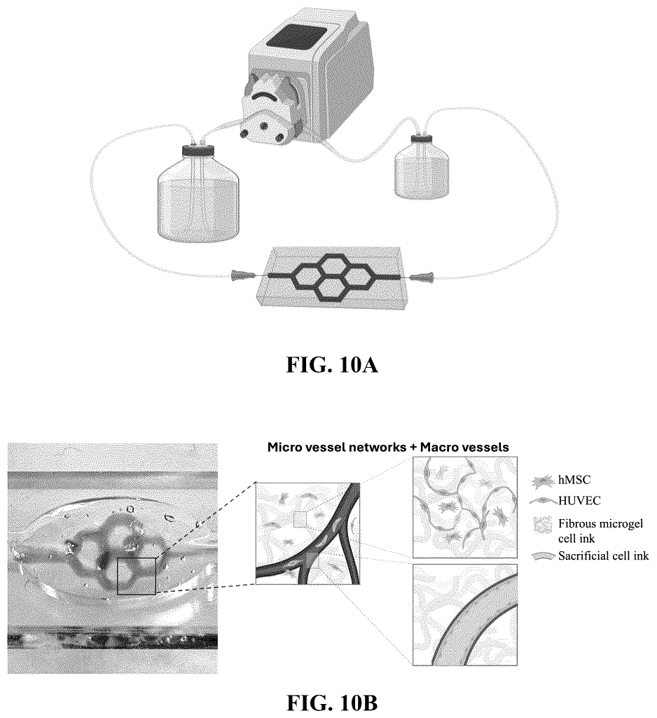

As illustrated in FIGS. 10A-10B, the fabrication process comprised the following steps:
(i) A custom-designed chamber composed of polydimethylsiloxane (PDMS) was filled with the MF bioink to form a physically stabilized support matrix;
(ii) A sacrificial bioink was extruded into the MF bath along a pre-programmed three-dimensional trajectory configured to form a closed-loop macrovascular circuit, consisting of two terminal perfusion ports and four interconnected hexagonal units simulating a hepatic lobule vasculature;
(iii) Following sacrificial ink deposition, the MF matrix was crosslinked via photopolymerization to stabilize the construct; and
(iv) The sacrificial bioink was removed under aqueous or thermal conditions to yield open, perfusable macrochannels, wherein perfusion needles were inserted at inlet/outlet ports to enable external fluid access.

This fabrication process resulted in the formation of a hierarchical vascular system, comprising: (i) engineered macro-channels enabling convective perfusion and (ii) MF-based microporous domains supporting localized diffusion and cell-cell communication. As shown in FIG. 10B, this architecture establishes a functionally integrated, multi-scale circulatory network within the construct.

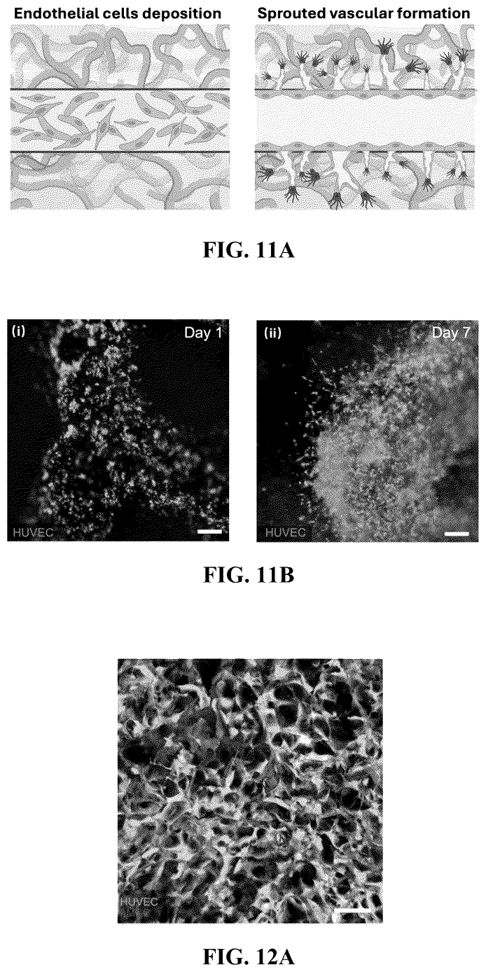

To evaluate the endothelialization capability of the engineered macrochannels, a sacrificial gelatin-based bioink containing human umbilical vein endothelial cells (HUVECs) was printed into an acellular MF bath. Upon liquefaction of the sacrificial ink at physiological temperature (37° C.), HUVECs were released into the lumen of the newly formed channels (FIG. 11A).

Fluorescence microscopy at day 1 confirmed uniform adhesion of HUVECs along the channel interior (FIG. 11B(i)). By day 7, HUVECs demonstrated proliferation and directional sprouting into the surrounding MF matrix, forming radially aligned angiogenic structures (FIG. 11B(ii)). These results indicate that the suspension-printed channels not only support in situ cell deposition but also promote functional endothelialization and cellular interface integration.

In a separate embodiment, the MF matrix was preloaded with a co-culture of HUVECs and human mesenchymal stem cells (hMSCs) to evaluate its ability to support spontaneous microvascular organization.

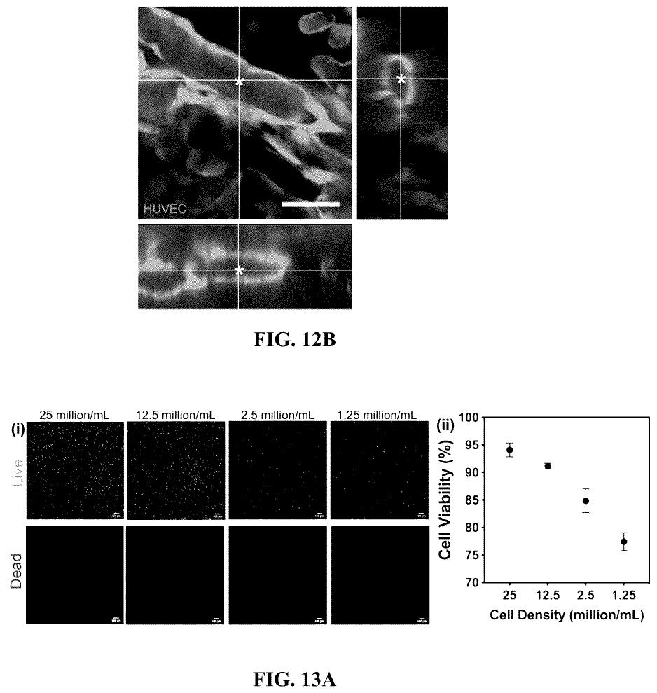

As shown in FIG. 12A, by day 10 of in vitro incubation, endothelial cells formed an interconnected, reticulated capillary plexus throughout the MF scaffold. By day 14, further network maturation was observed, including formation of hollow endothelial lumens, thereby confirming the establishment of physiologically relevant capillary-like microvessels (FIG. 12B). Collectively, these results validate our suspension printing approach as a powerful platform for fabricating multi-scale vascularized tissues, successfully combining engineered, perfusable vascular channels with self-assembled micro-capillaries.

### Example 6

### Structurally Guided Cellular Patterning Using Fibrous Microgel

### Example 6A

This example demonstrates the application of the disclosed dual-bioink bioprinting platform for the fabrication of engineered tissue constructs comprising one or more cell types distributed in spatially defined architectures. In particular, the method enables high-density cell encapsulation, void channel formation, and cell-to-cell interactions within the printed matrix and sacrificial regions.

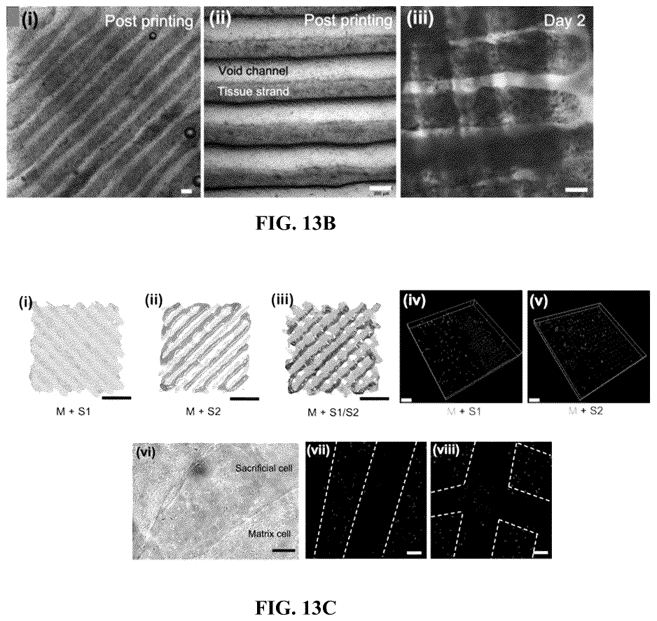

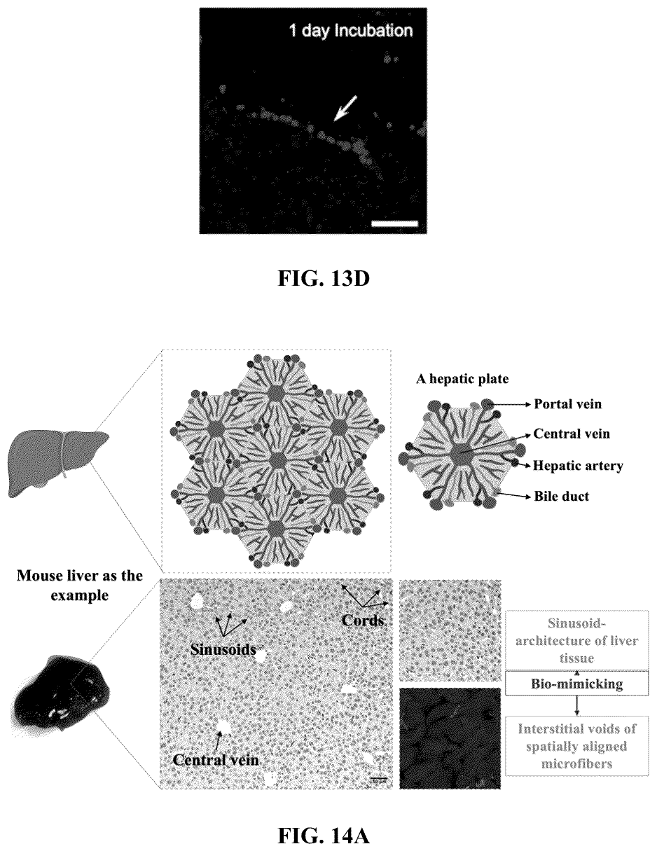

In one embodiment, the cell-laden matrix inks are compatible with cell densities ranging from 1.25 to 12.5 million cells per mL, with observed cell viability up to 94% at higher densities (FIG. 13A). Single-cell type tissue constructs were created using dual-bioinks with a cross-printing pattern (FIG. 7A). Upon removal of the sacrificial inks, void channels formed and cell diffusion from the matrix inks was observed (FIG. 13B(i) and (ii)). After 48 hours of culture, the cells in the matrix formed compact structures (FIG. 13B(iii)). These features may contribute to nutrient exchange and oxygen diffusion within the engineered tissue.

In another embodiment, multi-cellular constructs were fabricated by co-printing at least one matrix bioink (M) and two distinct sacrificial bioinks, each loaded with a different cell population. An interlaced deposition pattern was employed (FIG. 13C). After sacrificial ink removal and 24 hours of incubation, cells from the sacrificial inks aligned along the matrix filament surfaces, forming luminal structures with adherent cells outlining the channels (FIG. 13D). This technique enables spatial control over cell type localization and interaction dynamics, critical for modeling complex tissue microenvironments.

### Example 6B

In one embodiment, the structural guidance capacity of the fibrous microgel was evaluated for its ability to replicate native liver sinusoid-like microarchitecture, in the absence of predefined macrovascular channels or patterning cues.

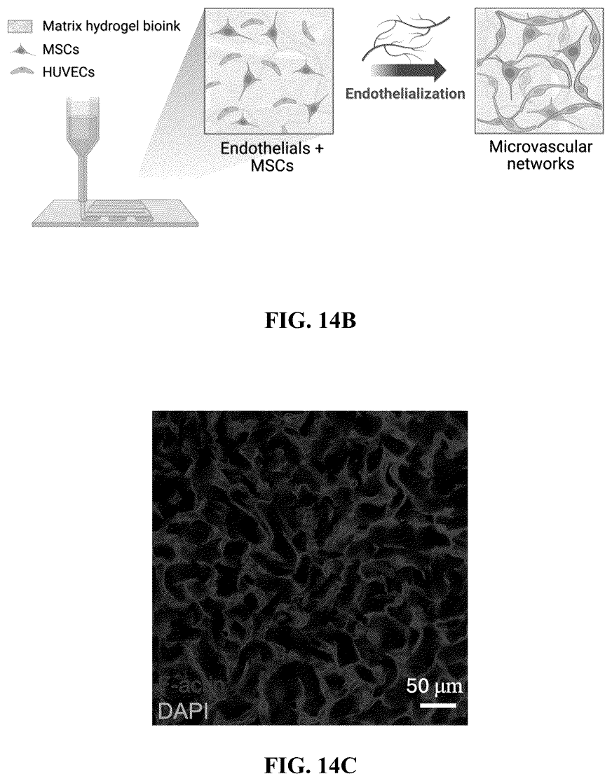

Histological analysis of murine liver revealed a densely branched, sinusoidal network defined by narrow interstitial spaces between hepatocyte plates (FIG. 14A). It was hypothesized that the interconnected microporous architecture of the fibrous microgel, comprising hydrogel filaments within the 20-50 μm diameter range, could serve as a physical scaffold to direct vascular cell self-organization into biomimetic sinusoid-like morphologies.

To test this hypothesis, a co-culture of human umbilical vein endothelial cells (HUVECs) and human mesenchymal stem cells (hMSCs) was homogeneously mixed into the fibrous microgel bioink to form a cell-laden matrix without any printed vascular pattern. The construct was cultured under standard in vitro conditions for 14 days.

Confocal microscopy imaging revealed that the embedded cells elongated along the microgel filaments and self-organized into a continuous, web-like capillary network. The cellular structures followed the topographical guidance of the fibrous framework, wrapping around microgel strands and forming a branched architecture throughout the matrix (FIGS. 14B-14C).

The resulting morphology recapitulated key features of liver sinusoids, including lumenized interstitial spacing and aligned capillary branches. These findings demonstrate that the fibrous microgel is not merely a permissive scaffold but functions as an active topographical instructive environment. Such structural biomimicry is of significant value for tissue-specific engineering of hepatic and other sinusoid-rich organs.

### Example 7

### Bioengineering Capillary Networks Through Co-Culture in Microfibrous Hydrogels

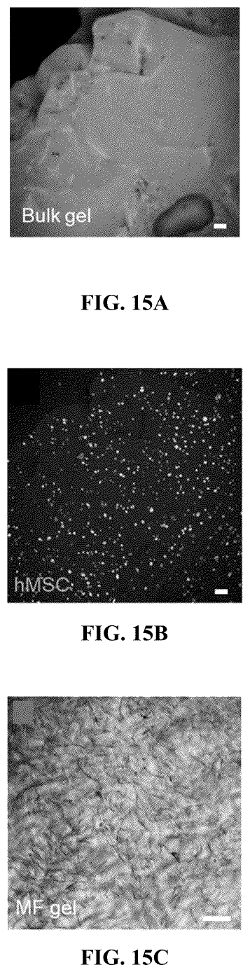

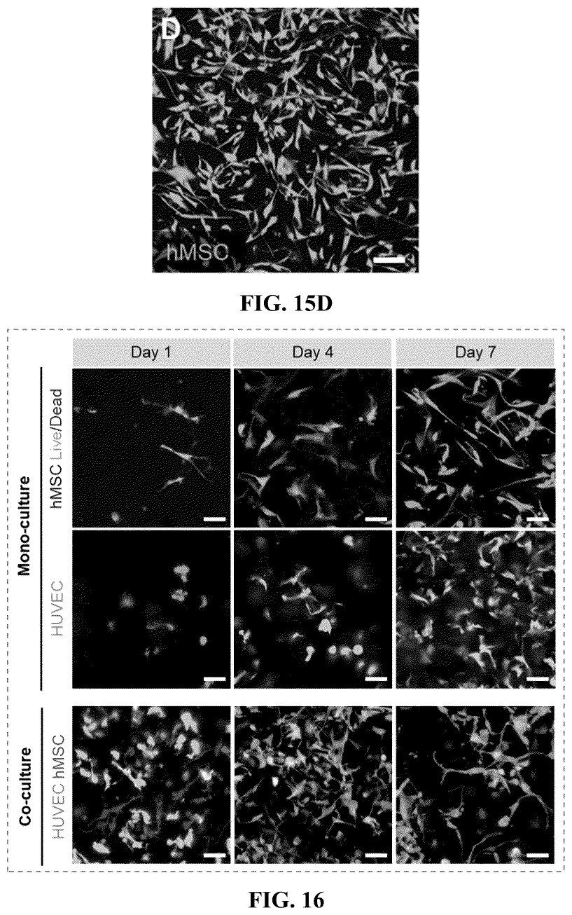

This bioprinting method includes the use of specialized matrix bioinks. A microfibrous matrix cell ink was explored for vascular network formation. Endothelial cells were encapsulated into microfibrous structural bioinks. FIGS. 15A-15D show that cells in microfibrous bioinks maintain an elongated, stretched morphology compared to their rounded shape in bulk gels.

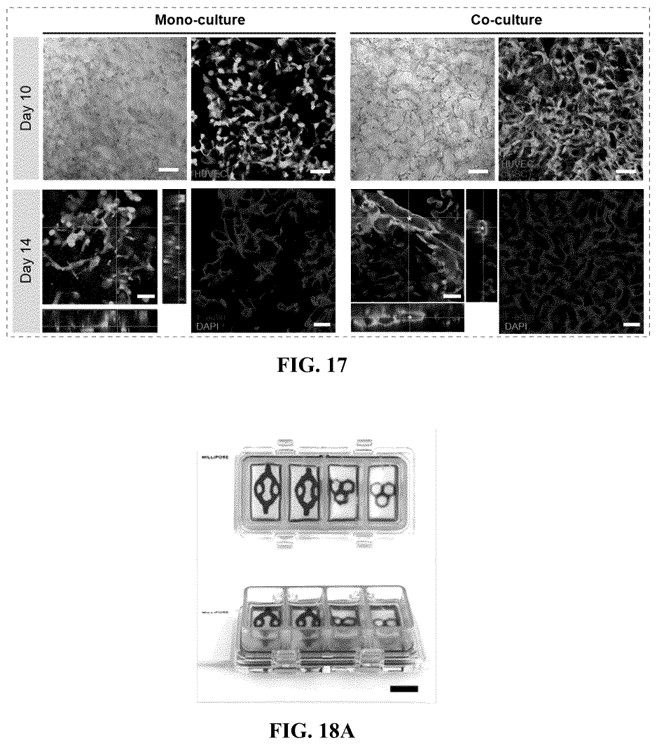

A co-culture system of HUVECs and hMSCs at a 1:1 ratio was employed. Observations over a 14-day culture period (FIGS. 16 and 17) indicated cell spreading and morphology in co-culture conditions. By day 7, co-cultured endothelial cells began forming sprout structures. This continued with extended culture, resulting in an interconnected micro-capillary network by day 10. By day 14, the presence of hollow endothelial lumens confirmed the successful formation of a functional microvascular network. These results validate the microfibrous matrix as a bioactive scaffold capable of guiding microvascular morphogenesis via cell-intrinsic mechanisms.

### Example 8

### In-Situ Development of Macrovascular Structures Using HUVEC-Laden Sacrificial Bioink

This example illustrates a dual-compartment vascularization approach wherein large perfusable channels are generated via a time-delayed, sacrificial bioink loaded with endothelial cells.

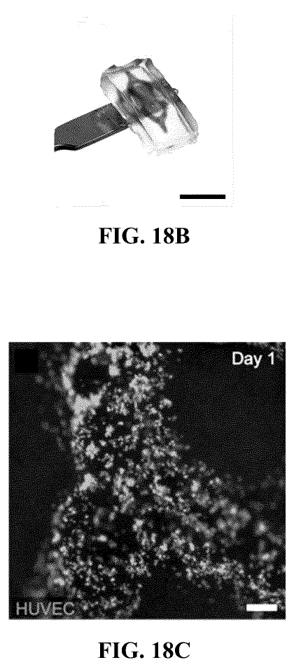

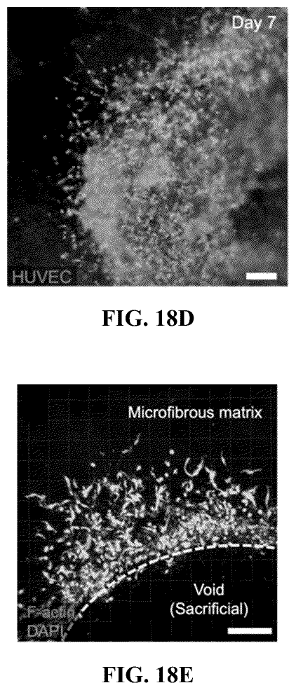

This method integrates microfibrous matrix inks with a time-delayed sacrificial bioink (FIG. 6A). The platform allows for the production of vascularized tissue constructs, as demonstrated by printing in a 4-well cell culture chamber (FIG. 18A). Following printing, the constructs undergo a second photopolymerization (FIG. 18B).

To elucidate the vascularization process, cell-free microfibrous ink and HUVEC-encapsulated sacrificial ink were used. 7.5% gelatin was used as the sacrificial material. Initially, as the gelatin dissolves, endothelial cells form aggregate clusters and adhere to the matrix hydrogel surface (FIG. 18C). By day 7, these cells invade the surrounding matrix tissue and develop sprouts, elongating to form vascular structures (FIGS. 18D-18E).

This dual-bioink approach allows for the development of two distinct vascular components within the tissue construct: the microfibrous matrix supports capillary network formation, while the sacrificial gelatin ink enables the creation of larger channels lined with endothelial cells.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Industrial Applicability

The invention has wide-ranging industrial applications, particularly in the fields of tissue engineering, regenerative medicine, and basic research.

Its primary functions include the creation of complex 3D vascularized tissue constructs, offering precise control over cell distribution within engineered tissues and enabling the formation of multi-scale vascular networks, from capillaries to larger vessels. The invention supports high cell viability in bioprinted constructs and promotes effective nutrient exchange and oxygen diffusion within these tissues. In tissue engineering, it can be used to develop more complex and functional tissue constructs and create tissue models for various organ systems. In regenerative medicine, the invention holds potential for developing implantable tissue constructs. It is also useful for basic research, providing insights into vascularization processes, cell-cell, and cell-matrix interactions in 3D environments. Furthermore, its application in multi-cellular tissue engineering allows for the incorporation of multiple cell types in precise spatial arrangements, replicating complex tissue microenvironments.

What is claimed is:

1. A method for multi-cellular bioprinting of vascularized tissue constructs, comprising:
preparing a degradable matrix bioink comprising gelatin methacrylate (GelMA) and gelatin;
preparing a sacrificial bioink comprising a hydrogel-forming component and a calcium agent;
loading the matrix bioink and the sacrificial bioink into separate cartridges of a multi-nozzle three dimensional (3D) bioprinter;
co-printing the matrix bioink and the sacrificial bioink in an alternating or embedded pattern to form a 3D tissue construct;
crosslinking the matrix bioink and removing the sacrificial bioink to create perfusable channels within the construct; and
forming the vascularized tissue constructs comprising a degradable matrix and an integrated network of perfusable channels.

2. The method of claim 1, wherein the matrix bioink further comprises lithium phenyl-2,4,6-trimethylbenzoylphosphinate (LAP) as a photoinitiator.

3. The method of claim 1, wherein the matrix bioink comprises microporous fibrous microgel particles formed by fragmenting a crosslinked bulk hydrogel, and the fibrous microgel particles are prepared by extruding a crosslinked hydrogel through a 20 μm mesh.

4. The method of claim 1, wherein the sacrificial bioink comprises endothelial cells at a density in a range from 5×10⁵ to 2×10⁶ cells/mL, and wherein the cells adhere to the inner channel surfaces after sacrificial ink removal.

5. The method of claim 1, wherein the 3D tissue construct comprises a hexagonal macrochannel geometry mimicking native liver lobules.

6. The method of claim 1, wherein the sacrificial bioink is printed directly into a self-healing support bath comprising a fibrous microgel jammed microporous matrix,

7. The method of claim 6, wherein the printed macrochannels define a perfusable circuit comprising at least one inlet and at least one outlet.

8. A vascularized tissue construct for implantation into a host organ, comprising:
a microporous matrix with interconnected micropores formed from a bioink composition;
a perfusable network of macrochannels extending through the microporous matrix; and
one or more populations of living cells encapsulated within the microporous matrix, wherein the vascularized tissue construct exhibits hierarchical porosity defined by the macrochannels and the interconnected micropores of the microporous matrix; and
wherein the construct comprises a self-organized microvascular plexus formed within the microporous matrix.

9. The vascularized tissue construct of claim 8, wherein the bioink composition comprises a suspension of crosslinked hydrogel microparticles comprising GelMA and gelatin, wherein the suspension of crosslinked hydrogel microparticles form a jammed, microporous matrix with interconnected pores ranging from 20 to 50 μm, and wherein the matrix exhibits shear-thinning and self-healing thixotropic properties.

10. The vascularized tissue construct of claim 8, wherein the interconnected micropores are configured to guide vascular cells to form sinusoid-like capillary structures.

11. The vascularized tissue construct of claim 8, wherein the inner surfaces of the macrochannels are lined with endothelial cells that originate from a sacrificial ink.

12. The vascularized tissue construct of claim 8, wherein the encapsulated cells comprise a co-culture of human mesenchymal stem cells and endothelial cells, and wherein the endothelial cells self-organize into a branched capillary plexus along the interstitial voids of the microporous matrix, guided by the topographical features of the matrix.

13. The vascularized tissue construct of claim 8, wherein the perfusable network of macrochannels comprises at least one inlet and at least one outlet port, configured to permit bidirectional perfusion through the tissue construct.

14. The vascularized tissue construct of claim 8, wherein the microporous matrix mimics liver sinusoidal microarchitecture.

15. A system for bioprinting vascularized tissue constructs, comprising:
a programmable, multi-nozzle 3D bioprinter with at least two independently controlled printheads, at least one of the temperature-controlled cartridge dispensers is configured to dispense a gelatin-based sacrificial bioink at 20-30° C.;
one or more temperature-controlled cartridge dispensers for bioinks;
a photocrosslinking apparatus configured to deliver blue light; and
a chamber for holding a self-healing support bath during embedded or suspension printing.

16. The system of claim 15, further comprising a dual-syringe Y-connector for mixing a cell suspension into the matrix bioink under aseptic conditions.

17. The system of claim 15, further comprising nozzles with different gauge sizes for generating vascular structures with distinct channel diameters.

18. The system of claim 15, wherein at least one controlled printhead is pneumatic-pressure driven and digitally controlled to regulate extrusion.

19. The system of claim 15, further comprising a microfibrous bioink preparation apparatus including a nylon mesh of approximately 20.0 μm pore size and a planetary centrifugal mixer.

20. The system of claim 15, further comprising a PDMS well chamber configured to contain the self-healing support bath during suspension printing.
